(19) United States
(12) Patent Application Publication
(10) Pub. No.: US 2004/0045560 A1
(43) Pub. Date: Mar. 11, 2004

(54) Computer-assisted multi-dimensional patient-selection

(75) Inventors: David M. Kent, Cambridge, MA (US); Robin Ruthazer, Newton, MA (US); Harry P. Selker, Wellesley, MA (US)

Correspondence Address: HALE AND DORR, LLP, 60 STATE STREET, BOSTON, MA, 02109

(73) Assignee: New England Medical Center Hospitals, Inc.

(21) Appl. No.: 10/446,264

(22) Filed: May 23, 2003

Related U.S. Application Data
(60) Provisional application No. 60/382,770, filed on May 23, 2002.
(60) Provisional application No. 60/440,294, filed on Jan. 15, 2003.

(51) Int. Cl. A61B 19/00

(52) U.S. Cl. 128/897

(57) ABSTRACT

An instrument for use with a patient who is experiencing acute ischemic stroke, the instrument for indicating whether to administer thrombolytic therapy to the patient after a predetermined elapsed time since onset of symptoms has passed, the instrument including an input device through which a user enters clinical information about the patient who is experiencing acute ischemic stroke; and a processor module programmed to use the entered clinical information to compute a predicted benefit to the health of the patient as a consequence of administering thrombolytic therapy to the patient at an onset to treatment time (OTT) that is greater than a predetermined time, the predicted benefit being derived from the entered clinical data.

### CROSS-REFERENCE TO RELATED APPLICATIONS

This application claims benefit under 35 U.S.C. §119(e) of prior U.S. Provisional Application No. 60/382,770, entitled “Computer-Assisted Multi-Dimensional Patient Selection,” filed on May 23, 2002 and U.S. Provisional Application No. 60/440,294, entitled “Computer-Assisted Multi-Dimensional Patient Selection,” filed on Jan. 15, 2003, both of which are incorporated herein by reference.

### TECHNICAL AREA OF THE INVENTION

The invention generally relates to a method and apparatus for determining what population of patients should receive thrombolytic thereapy.

### BACKGROUND

Thrombolytic therapy (TT) was first tested as a treatment for acute ischemic stroke (AIS) over 40 years ago. Following more than a dozen randomized clinical trials, the National Institute of Neurological Disorders and Stroke (NINDS) trial, published in 1995 was the first (and only) trial of TT in AIS to unequivocally demonstrate the efficacy of this treatment. The NINDS Study demonstrated that intravenous (IV) thrombolytic therapy improves outcomes in acute ischemic stroke (AIS), when delivered within 3 hours of symptom onset. In the NINDS trials, patients with ischemic stroke were treated within 3 hours of symptom-onset with either 0.9 mg/kg of rt-PA (maximum dose <90 mg) or placebo. A consistent 11% to 15% absolute benefit was found across 4 different stroke scales favoring rt-PA despite an increase in the symptomatic intracerebral hemorrhage (ICH) rate (6.4% vs 0.6%; P<0.001). Based on these results, the Food and Drug Administration (FDA) approved rt-PA for this indication in June of 1996.

However, the time limitation greatly restricts the number of patients that are eligible for treatment. It has been estimated that, since FDA approval, less than five percent of all stroke patients (and possible even less the 2%) are actually receiving rt-PA. Consequently, another study, call the ATLANTIS study, was initiated specifically to investigate whether treatment could be extended beyond 3 hours after symptom onset to include patients treated up to 5 hours after symptom onset. This trial found no overall benefit for the use of rt-PA in patients treated between 3 and 5 hours. Similarly, other trials with longer therapeutic time-windows (e.g. ECASS 1 and ECASS 2) also demonstrated no overall benefit.

Thus, the revolution for acute stroke treatment promised by the introduction of TT has not been fully realized. Currently, less than 5% (and possibly even less than 2%) of all stroke patients are receiving rt-PA. Impediments to the routine use of rt-PA therapy include the concern over the risk of thrombolytic-related ICH and the very limited time-window in which it is efficacious.

### SUMMARY OF THE INVENTION

In general, in one aspect the invention features an instrument for use with a patient who is experiencing acute ischemic stroke, the instrument for indicating whether to administer thrombolytic therapy to the patient after a predetermined elapsed time since onset of symptoms has passed. The instrument includes an input device through which a user enters clinical information about the patient who is experiencing acute ischemic stroke; and a processor module programmed to use the entered clinical information to compute a predicted benefit to the health of the patient as a consequence of administering thrombolytic therapy to the patient at an onset to treatment time (OTT) that is greater than a predetermined time. The predicted benefit is derived from the entered clinical data.

Embodiments include one or more of the following features. The predetermined elapsed time equals about 3 hours. The processor module is programmed to compute the predicted benefit by computing a first probability of a good outcome and a second probability of a good outcome, the first probability being computed using an assumption that no thrombolytic therapy is applied and the second probability being computed using an assumption that thrombolytic therapy is applied. The processor module is programmed to use an empirically based mathematical model to compute the first and second probabilities of a good outcome, wherein the model is derived from data about patients who were experiencing acute ischemic stroke and to whom thrombolytic therapy was administered. More specifically, the processor module is programmed to use a regression model to compute the first and second probabilities (e.g. a multivariate logistic regression model). The processor module is also programmed to generate an indication to administer thrombolytic thereapy to the patient when the second computed probability exceeds the first computed probability by a threshold amount (e.g. a threshold amount equal to zero).

In general, in another aspect, the invention features a method for use with a patient experiencing acute ischemic stroke. The method is for determining whether to administer thrombolytic therapy to the patient after a predetermined elapsed time since onset of symptoms has passed. The method involves receiving clinical information about the patient experiencing acute ischemic stroke; with a computer, computing an expected benefit to the health of the patient as a consequence of assuming that thrombolytic therapy is administered to the patient at an onset to treatment time (OTT) that is greater than a predetermined time; and using the expected benefit to determine whether to recommend that thrombolytic therapy be administered to the patient. The method also involves computing the benefit based on the received clinical information.

Other embodiments of the invention include one or moer of the following features. The predetermined elapsed time equals about 3 hours. The computing of the predicted benefit involves computing a first probability of a good outcome and computing a second probability of a good outcome, the first probability being computed using an assumption that no thrombolytic therapy is applied and the second probability being computed using an assumption that thrombolytic therapy is applied. The computing also involves using an empirically based mathematical model to compute the first and second probabilities of a good outcome, wherein the model being derived from data about patients who were experiencing acute ischemic stroke and to whom thrombolytic therapy was administered. More specifically, the computing involves using a regression model to compute the first and second probabilities (e.g. a multivariate logistic regression model). The method also involves generating an indication to administer thrombolytic thereapy to the patient when the second computed probability exceeds the first computed probability by a threshold amount (e.g. a threshold amount equal to zero).

A “multi-dimensional” scoring system, such as that mentioned above, has distinct advantages over conventional inclusion and exclusion criteria for selecting patients likely and unlikely to benefit. Such a scoring system potentially allows the exclusion of low-benefit/high-risk patients, while preserving a patient population with a favorable risk-benefit profile, even where such a population can not be identified using conventional criteria.

Further, in this case when statistical power is limited, independently-derived multidimensional variables (such as “risk of ICH”) have advantages over conventional “uni-dimensional” clinical variables (such as age, gender, etc.) when seeking variables that significantly interact with treatment effect. That is, even though “uni-dimensional” variables, such as age, blood pressure or the presence/absence of diabetes do not significantly interact with treatment effect, the well-developed and carefully selected “multi-dimensional” composite variable described herein, which combines these clinical variables into a weighted score, does interact with treatment effect.

These mathematical models could presumably be used for both the clinical trial and then for patient selection for treatment following the clinical trial. Software supporting the necessary computations could be incorporated into a wide-variety of devices, which might include desk-top computers in clinical workstations, hand-held devices (such as personal digital assistant) or localized medical devices (such as electrocardiographs or echocardiographs).

Patients likely to benefit from thrombolytic therapy even when treated after 3-hours from symptom-onset can be selected on the basis of easily obtainable, pre-treatment clinical information. If applied to patients that fall within the 3 to 6 hour window, this could potentially triple the number of patients eligible for thrombolytic therapy.

Other features and advantages of the invention will be apparent from the following detailed description and from the claims.

### BRIEF DESCRIPTION OF THE DRAWINGS

FIG. 1 presents a table of the variables and coefficients for the logistic-regression equation determines whether to administer thrombolytic therapy to patients who are experiencing acute ischemic stroke and whose onset symptoms occurred at least 3 hours earlier.

FIG. 2 is a schematic block diagram of a system that implements the algorithm described herein.

FIG. 3 is a flow chart showing the operation of the program for determining whether the administer thrombolytic therapy to the patient.

### DETAILED DESCRIPTION

Active medical therapies frequently have both benefits and risks. When clinical trials find that an active treatment is of no benefit overall, often this indicates that the marginal benefits of the therapy are, on average, negated by the risks of the therapy in the particular trial population overall. We recognized that such a result may obscure the fact that some patients within the trial benefit from therapy, while others are harmed. Thus, finding benefit or harm may depend on the proper selection of patients. This may be true even when conventional subgroup analyses, for example, those based on dichotomizing selected clinical variables, fail to find any significant differences between patient subgroups.

Patient selection in clinical trials is typically accomplished by identifying the variables that may influence the risks and benefits of therapy and setting boundary parameters (including/exclusion criteria) for each of these variables for patients with the index condition. Restricting patients by age and by co-morbid conditions, or designating cut-off values for clinical parameters such as blood pressure, would be the conventional approach to determining eligibility for a clinical trial. In such a conventional approach, each of the inclusion and exclusion criterion is used independent of the others.

In distinction to this, we elected to use of “multi-dimensional” scores, which simultaneously sum up the important clinical variables that influence the benefits and risks of therapy to select the most favorable population. We hypothesized that the absence in overall benefit in patients treated after 3 hours obscures the fact that a subset of patients may benefit from treatment, while another subset may be harmed. So, we developed a scoring system which would allow the selection of patients who are likely to benefit from thrombolytic therapy even when treated between 3 and 5 hours after symptom onset, based on easily obtainable clinical variables. Initially, to prove the concept, the score was based in part on extant clinical prediction models, which determine a patient's risk of thrombolytic-related intracranial hemorrhage.

Specifically, we hypothesized that an index derived on a database of patients treated with thrombolytic therapy for acute myocardial infarction identifying those likely to have a thrombolytic-related ICH may also identify patients more likely to have a thrombolytic-related ICH when treated for acute ischemic stroke and less likely to benefit from thrombolytic therapy for acute ischemic stroke. Patients at lower risk may be more likely to benefit, even after 3 hours.

We found that variables predictive of thrombolytic-related intercranial hemorrhage (ICH) risk are also predictive of risk in acute ischemic stroke but they not able to identify patients who do not benefit from rt-PA therapy though it was believed that the low ICH-risk patients mith be especially likely to benefit from therapy.

We then combined several randomized clinical trials into a large database, including NINDS 1 and 2, ATLANTIS A, ATLANTIS B, and ECASS 2 to develop a more sophisticated model. From this data, we constructed a derivation dataset and a test dataset. We used the derivation dataset to develop logistic regression models that predict the likelihood of a normal or near-normal outcome (mRS<=1), with and without thrombolytic therapy, using easily obtainable pre-treatment clinical characteristics.

These predictive models were then used on the independent test data to classify the subgroup of patients treated after 3 hours into “treatment-favorable” and “treatment-unfavorable” categories, based on their pre-treatment characteristics.

We then examined the actual outcomes in these subgroups with the hypothesis that patients classified by the model as “treatment unfavorable” would do worse with rt-PA than with placebo, while patients classified as “treatment-favorable” would benefit from thrombolytic therapy.

We examined the average outcomes in the “treatment unfavorable” group and the “treatment favorable group.” When the models were applied directly on the data on which they were developed, on average ⅓ of patients were classified as treatment-unfavorable and fully ⅔ of patients treated after 3 hours were classified as treatment favorable. Within the ⅓ of treatment unfavorable patients, outcomes were considerably worse with thrombolytic therapy than they were without thrombolytic therapy, by approximately an 11 point margin. Within the ⅔ of patients classified by the model as “treatment favorable”, on the other hand, outcomes were considerably better in the treated compared to the untreated group. There was an 8 percentage point margin of benefit.

Because the scoring system required a complex equation based on multiple patient characteristics, software supporting the score computation was incorporated into a computing platform. The computed score indicates whether the patient should be treated only within 3 hours of symptom onset, or whether the patient falls into the subgroup of patients who may get benefit with greater delays (e.g. up to 4 or 5 hours, depending on patient characteristics).

The Logistic Regression-Based Equation

The described embodiment employs a logistic regression-based equation for computing two probabilities or likelihoods that the patient will experience a good result. One computed probability assumes TT is not administered and the second computed probability assumes that TT is administered within a specified time after onset of the stroke symptoms.

The logistic regression equations are of the following general form:

$$P = 100 [ 1 - \frac{1}{1 + {ⅇ}^{z}} ] \qquad Eq . 1$$

where P is the probability of a particular medical outcome or diagnosis, and where z has the following general form:

z=b₀+Σbᵢxᵢ Eq. 2

In this equation, b₀ is a constant, and the bᵢ's are coefficients of the independent variables xᵢ which are included in the model.

Standard, well known regression techniques and other mathematical modeling were employed to identify the most appropriate set of independent variables, namely, the xᵢ 's, and to determine the values of the coefficients of these variables. For a description of such techniques and examples of commercially available computer programs that implement them, see N. C. Cary in SUGI Supplement Library User's Guide, SAS Institute, p. 181-202, 1983, and L. Engelman, “PLR Stepwise Logistic Regression,” BMDP Statistical Software, Chap. 14.5, pp. 330-334, BMDP publishers, Westwood, Calif.

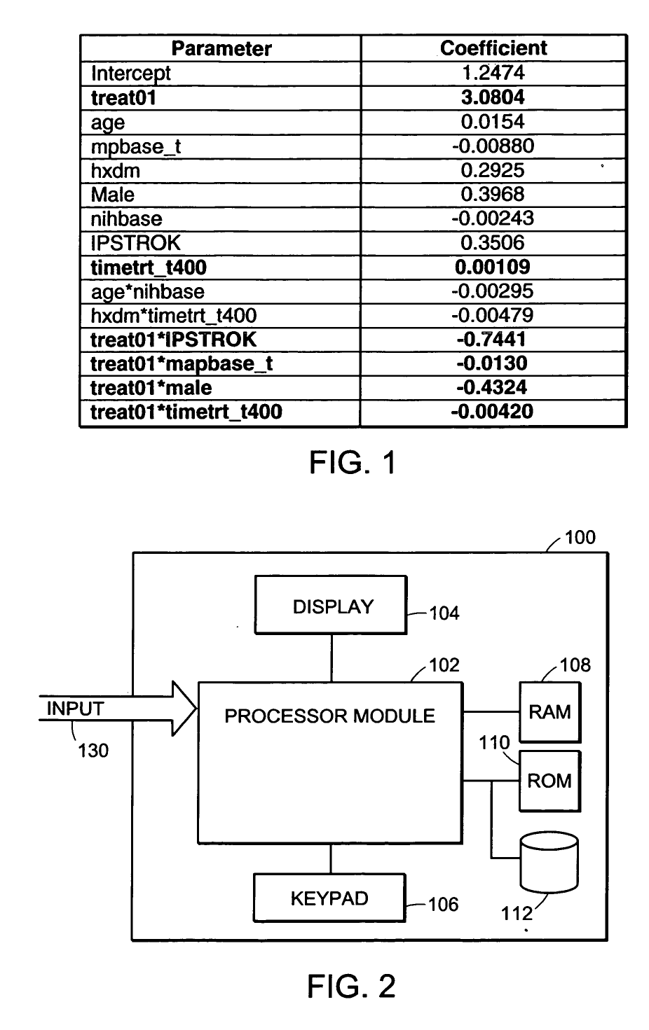

FIG. 1 shows a specific embodiment of Eq. 2 that was derived from available data. The equation computes a likelihood of a good outcome at 90 days after stroke onset. More specifically, it computes a measure of the outcome based on the well-known modified Rankin score (mRS). The modified Rankin Score is a score the value of which ranges from 0 to 6 (i.e., 0 for a normal, 1 for near normal, 2 for mild disability, 4 for severe disability, and 6 for dead).

The variables presented in FIG. 1 have the following meaning:

| | |
|---|---|
| treat01 | an indicator of whether rt-PA treatment is administered. |
| | This variable takes on two values, namely, 1 if it is |
| | assumed that rt-PA treatment (rt-PA) |
| | is to be administered and 0 if it is assumed that will |
| | not be administered; |
| age | age in years; |
| mapbase_t | mean arterial blood pressure; |
| hxdm | history of diabetes; |
| male | gender (male equals 1 and female equals 0); |
| nihbase | severity of presenting neurological deficit as measured |
| | by the National Institutes of Health Stroke Scale |
| | (NIHSS) score; |
| IPSTROK | indication of prior stroke; and |
| timetrt_t400 | onset to treatment time (OTT) measured in minutes. |

The equation also includes two interaction terms, namely, age*nihbase and hxdm*timetrt_t400, in which the two specified variables are multiplied together to generate the value for that variable.

There are also four variables that interact with treatment, namely, gender, prior stroke, MAP and symptom onset to treatment time. Those interactions are represented by the last four variables in the table shown in FIG. 1.

The coefficients, bᵢ, of the respective variables are presented in the right hand column of FIG. 1.

The equations yield a prediction of, for any patient, the likelihood of a good outcome with and without thrombolytic therapy. If the difference between these values is positive, then the patient is categorized as “treatment favorable”, if the difference is negative then the patient is categorized as “treatment unfavorable.” More specifically, the equations are used to compute two likelihoods, one assuming that TT will be administered at some time, timetrt_{—}400, after the onset of symptoms (L_{TT}) and the other one assuming that no TT is administered (L_{No}_{_{—}}_{TT}). If the result computed under the assumption that TT is administered exceeds the result computed under the assumption that TT is not administered by some threshold amount (i.e., if L_{TT}−L_{No}_{_{—}}_{TT}≧Threshold), then the indication is to administer the TT to the patient at least before the elapsed time since onset of symptoms exceeds timetrt_{—}400. In the described embodiment, the threshold is set to zero though it may be appropriate to select another threshold value that is a positive, non-zero value.

There are other variables that could be included in the model that might improve its performance. These other variables might substitute for existing variables or be in addition to them. They include, for example, radiologic variables, serum markers (such as TAFI or fibronectin), and a variable capturing CT scan information (e.g. findings of edema or mass effect) which is automatically obtained for patients who appear to be experiencing AIS.

Of course, the precise set of variables that are identified and the predictive ability of the resulting logistic equation generally depends upon the quality of the underlying data that is used to develop the model. Such factors as the size and completeness of the database are often of significant importance. The selection of the relevant variables and the computation of the appropriate coefficients are well within the skill of an ordinary person skilled in the art.

For examples of other predictive instruments which also use logistic-regression equations to compute probabilities of medical outcomes (e.g. the probability of ACI) refer to the following patents, all of which are incorporated herein by reference: U.S. Pat. No. 4,957,115 by Selker, entitled Device for Determining the Probability of Death of Cardiac Patients”; U.S. Pat. No. 4,998,535 by Selker et al., entitled “Thrombolysis Predictive Instrument”; U.S. Pat. No. 5,277,188 by Selker, entitled “Clinical Information Reporting System”; and U.S. Pat. Nos. 5,501,229, 5,718,233 and U.S. Pat. No. 5,724,983, all three of which are by Selker et al., and are entitled “Continuous Monitoring Using A Predictive Instrument”.

The Platform

The algorithm for determining whether to administer TT to the patient experiencing acute ischemic stroke (i.e., the predictive device) can be implemented on any platform that has adequate computing capabilities, e.g. a laptop computer 100 such as is schematically depicted FIG. 2. Alternatively, one might employ desktop computers within emergency departments or nursing stations, hand held computer devices such as personal digital assistants (PDAs), or localized medical devices such as electrocardiographs or CAT scans.

In general, such a computing platform will include a processor module 102 that has one or more microprocessors, a display device 104 such as a video monitor, and a conventional input device 106 such as a keyboard or keypad. The computing platform will also typically include associated memory such as, for example, random access memory (RAM) 108, read only memory (ROM) 110, and possibly other non-volatile memory such as disk memory 112. ROM and/or non-volatile memory stores the programs for computing the results of the logistic regression equation from the patient-related data entered by the user through input device 106. Laptop 100 might also include an input interface 130 for automatically receiving data from other patient monitoring equipment such as a blood pressure monitor (not shown).

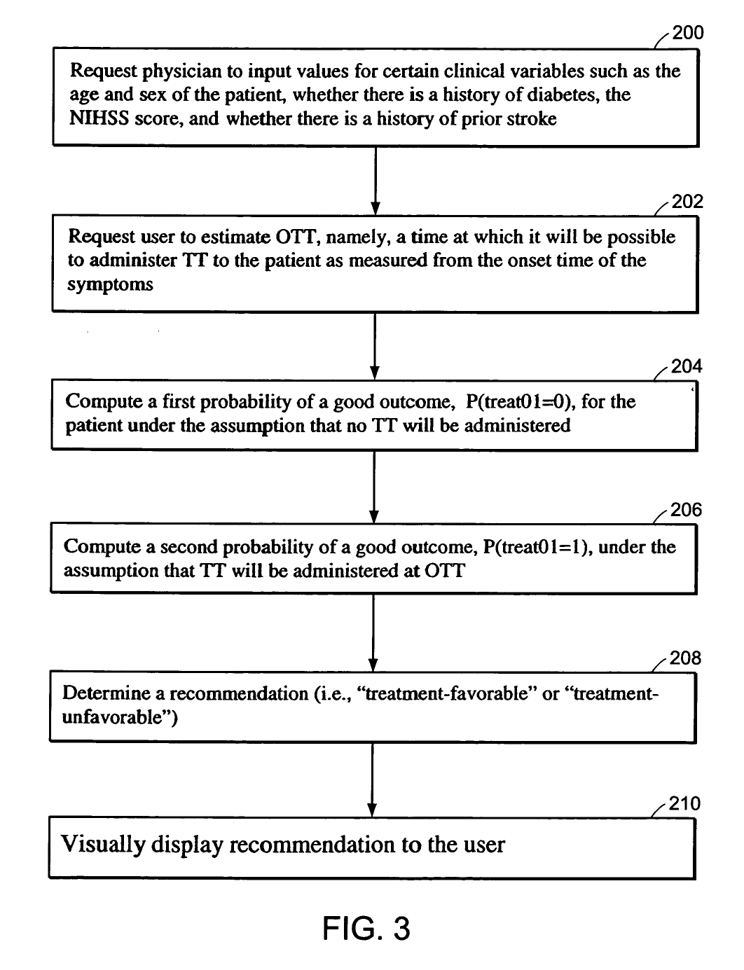

The predictive device operates as shown generally in FIG. 3. When the program that computes the algorithm is run on the laptop, it asks the physician to input values for certain clinical variables such as the age and sex of the patient, whether there is a history of diabetes, the NIHSS score, and whether there is a history of prior stroke (step 200). The request for this user input is in the form of a menu that is displayed on screen and that lists the variables for which inputs are desired. After the user has entered the required information, the program also requires the user to estimate OTT, namely, a time at which it will be possible to administer TT to the patient as measured from the onset time of the symptoms (step 202).

Once the program has received all of the input values necessary to compute Eq. 1, it then computes a first probability of a good outcome, P(treat01=0), for the patient under the assumption that no TT will be administered (step 204). Then, program computes a second probability of a good outcome, P(treat01=1), under the assumption that TT will be administered at OTT (step 206). In other words, the program computes Eq. 1 twice, first setting the value of variable treat01 equal to zero and then setting the value of variable treat01 equal to one.

After the program has computed the two probabilities of a good outcome, it uses those computed values to determine a recommendation (i.e., “treatment-favorable” or “treatment-unfavorable”) (step 208) and visually displays that to the user (step 210).

The program just described is stored on a computer readable medium such as a floppy disk or CD-ROM from which it can be loaded into RAM or ROM in the computer.

Other embodiments are within the following claims.

We claim:

1. An instrument for use with a patient who is experiencing acute ischemic stroke, the instrument for indicating whether to administer thrombolytic therapy to the patient after a predetermined elapsed time since onset of symptoms has passed, the instrument comprising:
an input device through which a user enters clinical information about the patient who is experiencing acute ischemic stroke; and
a processor module programmed to use said clinical information to compute a predicted benefit to the health of the patient as a consequence of administering thrombolytic therapy to said patient at an onset to treatment time (OTT) that is greater than a predetermined time, said predicted benefit being derived from said clinical data.

2. The instrument of claim 1 wherein the predetermined elapsed time equals about 3 hours.

3. The instrument of claim 2 wherein said processor module is programmed to compute said predicted benefit by computing a first probability of a good outcome and a second probability of a good outcome, the first probability being computed using an assumption that no thrombolytic therapy is applied and the second probability being computed using an assumption that thrombolytic therapy is applied.

4. The instrument of claim 3 wherein said processor module is programmed to use an empirically based mathematical model to compute the first and second probabilities of a good outcome, said model being derived from data about patients who were experiencing acute ischemic stroke and to whom thrombolytic therapy was administered.

5. The instrument of claim 4 wherein said processor module is programmed to use a regression model to compute the first and second probabilities.

6. The instrument of claim 5 wherein said processor module is programmed to use a multivariate logistic regression model to compute the first and second probabilities.

7. The instrument of claim 3 wherein said processor module is programmed to generate an indication to administer thrombolytic thereapy to said patient when said second computed probability exceeds the first computed probability by a threshold amount.

8. The instrument of claim 3 wherein said threshold amount equals zero.

9. The instrument of claim 1 wherein said processor module is programmed to compute said predicted benefit by computing a first probability of a good outcome and a second probability of a good outcome, the first probability being computed using an assumption that no thrombolytic therapy is applied and the second probability being computed using an assumption that thrombolytic therapy is applied.

10. The instrument of claim 9 wherein said processor module uses an empirically based mathematical model to compute the first and second probabilities, said model being derived from data about patients to whom thrombolytic therapy was administered.

11. The instrument of claim 10 wherein said processor module uses a regression model to compute the first and second probabilities.

12. The instrument of claim 11 wherein said processor module uses a multivariate logistic regression model to compute the first and second probabilities of acute hospital mortality.

13. A method for use with a patient experiencing acute ischemic stroke, said method for determining whether to administer thrombolytic therapy to said patient after a predetermined elapsed time since onset of symptoms has passed, the method comprising:
receiving clinical information about the patient experiencing acute ischemic stroke;
with a computer, computing an expected benefit to the health of the patient as a consequence of assuming that thrombolytic therapy is administered to said patient at an onset to treatment time (OTT) that is greater than a predetermined time, said computed benefit being derived from the received clinical information; and
using the expected benefit to determine whether to recommend that thrombolytic therapy be administered to said patient.

14. The method of claim 13 wherein the predetermined elapsed time equals about 3 hours.

15. The method of claim 14 wherein the computing of said predicted benefit involves computing a first probability of a good outcome and computing a second probability of a good outcome, the first probability being computed using an assumption that no thrombolytic therapy is applied and the second probability being computed using an assumption that thrombolytic therapy is applied.

16. The method of claim 15 wherein said computing involves using an empirically based mathematical model to compute the first and second probabilities of a good outcome, said model being derived from data about patients who were experiencing acute ischemic stroke and to whom thrombolytic therapy was administered.

17. The method of claim 15 wherein said computing involves using a regression model to compute the first and second probabilities.

18. The method of claim 15 wherein said computing involves using a multivariate logistic regression model to compute the first and second probabilities.

19. The method of claim 14 further comprising generating an indication to administer thrombolytic thereapy to said patient when said second computed probability exceeds the first computed probability by a threshold amount.

20. The method of claim 19 wherein said threshold amount equals zero.
